(19) United States
(12) Patent Application Publication
(10) Pub. No.: US 2013/0309189 A1
(43) Pub. Date: Nov. 21, 2013

(54) NON-WASHING COSMETIC COMPOSITION COMPRISING AT LEAST ONE IONIC FIXING POLYMER AND AT LEAST ONE ESTER OF POLYETHYLENE GLYCOL AND OF FATTY ACID, AND METHOD FOR FIXING A HAIRSTYLE

(71) Applicant: L'OREAL, Paris (FR)

(72) Inventors: Sabine VRIGNAUD, la Plaine (FR); Cécile BEBOT, Clichy (FR)

(73) Assignee: L'OREAL, Paris (FR)

(21) Appl. No.: 13/942,195

(22) Filed: Jul. 15, 2013

Related U.S. Application Data
(62) Division of application No. 11/655,861, filed on Jan. 22, 2007, now abandoned.
(60) Provisional application No. 60/771,039, filed on Feb. 8, 2006.

(30) Foreign Application Priority Data
Jan. 20, 2006 (FR) 06 00559

(51) Int. Cl. A61K 8/86; A61K 8/81; A61Q 5/06

(52) U.S. Cl. CPC A61K 8/86; A61Q 5/06; A61K 8/8158; A61K 8/817; A61K 8/8152
USPC 424/70.6; 424/70.9; 424/70.13; 424/70.16; 424/70.17; 424/70.12

(57) ABSTRACT

Disclosed herein is a non-washing cosmetic composition comprising, in a cosmetically acceptable medium chosen from aqueous/alcoholic and alcoholic mediums, at least one ionic fixing polymer, at least one ester of polyethylene glycol and of fatty acid, and at least one thickening agent. Also disclosed herein is a process for the shaping and/or the form retention of a hairstyle comprising applying this cosmetic composition to the hair.

This application claims benefit of U.S. Provisional Application No. 60/771,039, filed Feb. 8, 2006, the contents of which are incorporated herein by reference. This application also claims benefit of priority under 35 U.S.C. §119 to French Patent Application No. FR 06 00559, filed Jan. 20, 2006, the contents of which are also incorporated herein by reference.

Disclosed herein is a non-washing cosmetic composition. Also disclosed herein is a method for fixing keratinous fibers, for example, human keratinous fibers, such as the hair.

The most common cosmetic compositions on the cosmetic market for hair shaping and/or form retention of the hairstyle are compositions comprising a solution, for example, a solution chosen from alcoholic and aqueous/alcoholic solutions, and at least one component, referred to as a fixing component, which generally may be chosen from polymeric resins, the role of which is to form joins between the hairs. The at least one fixing component is often formulated as a mixture with various cosmetic adjuvants.

The compositions may also be provided in the form of gels.

These cosmetic compositions may be packaged in various containers, for example, pots, tubes, pump-action spray containers, and appropriate aerosol containers pressurized using a propellant. The aerosol system may comprise, on the one hand, a liquid (or dispensable) phase and, on the other hand, a propellant.

Once on the hair, the composition comprising the at least one fixing component and the at least one appropriate solvent dries, making possible the formation of joins necessary for the fixing of the hair by the fixing components. The joins should be sufficiently rigid to provide the form retention of the hair; however, they should also be sufficiently weak for the user to be able, by combing or by brushing the hair, to destroy them without hurting the scalp and/or damaging the hair.

The conventional film-forming resins generally used as a fixing agent in an alcoholic medium can exhibit the disadvantage of conferring mediocre cosmetic properties on the styling composition; for instance, the feel obtained by the use of the compositions based on film-forming resins may not be very satisfactory.

Generally, the fixing polymers make it possible to indeed fix the hairstyle in the desired shape. However, during the day, the hairstyle may be subjected to various deformations (passing of the hand through the hair, wearing of a hood, and the like) which, little by little, can cause the polymer film to crumble and reduce the hold of the hairstyle.

Accordingly, there is a need in the art for cosmetic compositions that can provide good styling properties without the aforementioned drawbacks. The present inventors have discovered, surprisingly and unexpectedly, that the use of a combination of at least one ionic fixing polymer and at least one ester of polyethylene glycol and of fatty acid can make it possible to satisfactorily fix the hairstyle and also to confer on it a shape which can last longer than the hairstyles shaped using a conventional fixing composition.

These compositions can also make it possible to confer satisfactory cosmetic properties on the hair.

Other characteristics, aspects, objects, and benefits of the present disclosure will become even more clearly apparent upon reading the description and examples which follow.

Disclosed herein is a non-washing cosmetic composition comprising, in a cosmetically acceptable medium, at least one ionic fixing polymer and at least one ester of polyethylene glycol and of fatty acid.

In at least one embodiment, the cosmetic composition is a hair composition, for example, a hair styling cosmetic composition.

The cosmetic composition according to the present disclosure can be provided in a form chosen from sprays, foams, and gels.

Also disclosed herein is a process for the shaping or form retention of the hairstyle comprising applying a cosmetic composition according to the present disclosure to the hair.

Further disclosed herein is the use of a cosmetic composition of the present disclosure as styling composition for the fixing and form retention of the hair, for example, for conferring good hold over time on the hairstyle.

The compositions according to the present disclosure, in at least one embodiment, are non-washing (non-detergent) compositions; and may comprise less than 4% by weight of detergent surfactants, for instance, less than 1% by weight, with respect to the total weight of the composition, and in another embodiment, do not comprise detergent surfactants at all.

As used herein, the term “detergent surfactants” means any anionic or nonionic surface-active agent, different from the esters of polyethylene glycol and of fatty acid of the present disclosure.

The cosmetically acceptable medium used in the compositions according to the present invention may be chosen from aqueous/alcoholic mediums and alcoholic mediums.

The at least one alcohol used in the compositions according to the present disclosure can be chosen, for example, from monohydroxylated alcohols and polyols. In at least one embodiment, the monohydroxylated alcohol may be chosen from lower C₁-C₄ alcohols, such as ethanol, isopropanol, tert-butanol, and n-butanol. In another embodiment, the alcohol is ethanol.

Non-limiting examples of polyols which can be used in the compositions according to the present disclosure include propylene glycol, polyethylene glycols, polyol ethers, and mixtures thereof.

The at least one alcohol may be present in the composition according to the present disclosure in an amount ranging from 0.1 to 99%, for example, from 0.5 to 50%, or from 1 to 30% by weight, with respect to the total weight of the composition.

In another embodiment, the at least one alcohol may be present in the composition of the present disclosure in an amount ranging from 2 to 10% by weight, with respect to the total weight of the composition.

Esters of Polyethylene Glycol and of Fatty Acid

Any ester of polyethylene glycol and of fatty acid may be used in the compositions according to the present disclosure. These esters are capable of being obtained by esterification of at least one polyethylene glycol comprising at least two OCH₂CH₂ units optionally in combination with at least one unit chosen from:

—OCH₂—CH(CH₃)—,

—OCH₂—CH(OH)CH₂—, and

—CH₂—CH(CH₂OH)—O—

and of at least one saturated or unsaturated, linear or branched fatty acid comprising from 8 to 40, for example, from 8 to 30, carbon atoms. In at least one embodiment, the at least one fatty acid is saturated.

According to another embodiment, the ester of polyethylene glycol and of fatty acid is the ester of polyethylene glycol and of a fatty acid of the formula:

R₁CO—(OCH₂CH₂)ₙ₀-[OCH₂—CH(OR₂)—CH₂]ₙ₁—(OCH₂CH₂)ₙ₂—R₃

wherein:

R₂ is chosen from hydrogen and (CH₂CH₂O)ₙ₃COR₄ groups;

n1 is an integer equal to 0 or 1;

n2 is an integer ranging from 2 to 300;

n3 is an integer ranging from 1 to 300;

n0 is an integer ranging from 0 to 300;

R₃ is chosen from hydrogen, OH groups, and R₅COO groups; and

R₁, R₄, and R₅, which may be identical or different, are chosen from C₁₀ to C₃₀ alkyl groups and C₁₀ to C₃₀ alkylene groups.

Non-limiting examples of suitable esters according to the present disclosure include polyethylene glycol (150 EO) distearate and glyceryl oxyethylene (200 EO) monostearate.

The at least one ester of polyethylene glycol and of fatty acid may be present in the composition in an amount ranging from 0.01% to 20% by weight, for example, from 0.1% to 15% by weight, or from 1% to 10% by weight, with respect to the total weight of the composition.

Ionic Fixing Polymers

All the ionic fixing polymers, including anionic fixing polymers, cationic fixing polymers, amphoteric fixing polymers, and mixtures thereof known in the hair care field can be used in the compositions according to the present disclosure.

Examples of anionic polymers include, but are not limited to, polymers comprising groups derived from acids chosen from carboxylic, sulphonic, and phosphoric acids and exhibiting a weight-average molecular weight ranging from 500 to 5,000,000.

The carboxyl groups may be contributed by monomers chosen from unsaturated monocarboxylic acid and dicarboxylic acid monomers, such as those corresponding those of formula (I):

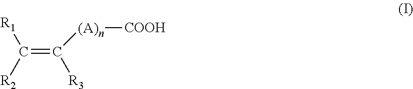

wherein:

n is an integer ranging from 0 to 10,

A is a methylene group, optionally connected to the carbon atom of the unsaturated group or to the neighboring methylene group, when n is greater than 1, via a heteroatom, such as oxygen and sulphur,

R₁ is chosen from hydrogen, phenyl groups, and benzyl groups,

R₂ is chosen from hydrogen, lower alkyl groups, and carboxyl groups, and

R₃ is chosen from hydrogen, lower alkyl groups, —CH₂—COOH, phenyl groups, and benzyl groups.

In at least one embodiment, in formula (I), a lower alkyl group may comprise from 1 to 4 carbon atoms, for example, the lower alkyl group may be chosen from methyl and ethyl groups.

Non-limiting examples of anionic fixing polymers comprising carboxyl or sulpho groups include:

A) Homo- or copolymers of acrylic or methacrylic acid and salts thereof, including copolymers of acrylic acid and of acrylamide and methacrylic acid/acrylic acid/ethyl acrylate/methylmethacrylate copolymers, for instance, Amerhold DR 25, sold by Amerchol, and sodium salts of polyhydroxycarboxylic acids; and methacrylic acid/ethyl acrylate copolymers, for example, those in an aqueous dispersion, such as Luviflex Soft and Luvimer MAE, sold by BASF.

B) Copolymers of acrylic or methacrylic acid with a monoethylenic monomer, such as ethylene, styrene, vinyl esters, and esters of acrylic or methacrylic acid, optionally grafted onto a polyalkylene glycol, such as polyethylene glycol, and optionally crosslinked;

Such polymers are disclosed, for example, in French Patent No. 1 222 944 and German Application No. 2 330 956, the copolymers of this type comprising, in their chain, at least one optionally N-alkylated and/or -hydroxyalkylated acrylamide unit, such as those disclosed, for instance, in Luxembourgian Patent Applications 75370 and 75371. Copolymers of acrylic acid and of C₁-C₄ alkyl methacrylate may also be used.

C) Copolymers derived from crotonic acid, such as those comprising, in their chain, vinyl acetate or propionate units and optionally other monomers, such as allyl or methallyl esters, vinyl ether or vinyl ester of a saturated, linear or branched, carboxylic acid comprising a long hydrocarbon chain, such as those comprising at least 5 carbon atoms, it being possible for these polymers optionally to be grafted and crosslinked, or alternatively a vinyl, allyl or methallyl ester of an α- or β-cyclic carboxylic acid;

Such polymers are disclosed, for example, in French Patent Nos. 1 222 944, 1 580 545, 2 265 782, 2 265 781, 1 564 110, and 2 439 798. Non-limiting examples of commercial products falling within this class include the Resins 28-29-30, 26-13-14, and 28-13-10 sold by National Starch.

Copolymers derived from crotonic acid may also be used, for example, crotonic acid/vinyl acetate/vinyl tert-butylbenzoate terpolymers, such as Mexomere PW supplied by Chimex.

D) Polymers derived from maleic, fumaric, and itaconic acids and anhydrides with vinyl esters, vinyl ethers, vinyl halides, phenylvinyl derivatives, and acrylic acid and its esters; these polymers optionally being esterified;

Such polymers are disclosed, for example, in U.S. Pat. Nos. 2,047,398, 2,723,248, and 2,102,113 and British Patent No. 839 805, for example, those sold under the names Gantrez® AN and ES by ISP.

Polymers also falling within this class may include, for example, copolymers of maleic, citraconic, and itaconic anhydrides and of an allyl or methallyl ester, optionally comprising an acrylamide or methacrylamide group, an α-olefin, acrylic or methacrylic ester, acrylic or methacrylic acids or vinylpyrrolidone in their chain; the anhydride functional groups being monoesterified or monoamidated. These polymers are, for example, disclosed in French Patent Nos. 2 350 384 and 2 357 241.

E) Polyacrylamides comprising carboxylate groups;

F) Polymers comprising sulpho groups. These polymers may be chosen from polymers comprising vinylsulphonic, styrenesulphonic, naphthalenesulphonic, acrylamidoalkylsulphonic, and sulphoisophthalate units;

These polymers can be chosen, for example, from:

salts of polyvinylsulphonic acid having a molecular weight ranging from 1,000 to 100,000, and copolymers with an unsaturated comonomer, such as acrylic or methacrylic acids and their esters, acrylamide or its derivatives, vinylethers, and vinylpyrrolidone;

salts of polystyrenesulphonic acid, for instance, the sodium salts, having a molecular weight of approximately 500 000 and approximately 100 000. These compounds are disclosed, for instance, in French Patent No. 2 198 719; and

salts of polyacrylamidosulphonic acids, such as those disclosed in U.S. Pat. No. 4,128,631;

G) grafted anionic silicone polymers; chosen, for example, from polymers with a nonsilicone organic backbone grafted by monomers comprising a polysiloxane, polymers with a polysiloxane backbone grafted by nonsilicone organic monomers, and mixtures thereof.

As used herein, the terms “silicone” and “polysiloxane” are mean, in conformity with what is generally accepted, any organosilicon polymer or oligomer comprising a branched or crosslinked, linear or cyclic structure of variable molecular weight obtained by polymerization and/or polycondensation of suitably functionalized silanes and comprising a repetition of main units in which the silicon atoms are connected to one another via oxygen atoms (siloxane bond ≡Si—O—Si≡), optionally substituted hydrocarbon radicals being directly bonded via a carbon atom to the said silicon atoms. The most common hydrocarbon radicals include, by way of non-limiting example, alkyl radicals, such as C₁-C₁₀ alkyl radicals, for instance, methyl radicals, fluoroalkyl radicals, aryl radicals, such as phenyl radicals, and alkenyl radicals, such as vinyl radicals; and other types of radicals capable of being bonded either directly or via a hydrocarbon radical to the siloxane chain, including hydrogen, halogens such as chlorine, bromine, and fluorine, thiols, alkoxy radicals, polyoxyalkylene (or polyether) radicals such as polyoxyethylene and/or polyoxypropylene radicals, hydroxyl radicals, hydroxyalkyl radicals, amino groups, which may or may not be substituted, amide groups, acyloxy radicals, acyloxyalkyl radicals, hydroxyalkylamino radicals, aminoalkyl radicals, quaternary ammonium groups, amphoteric groups, betaine groups, and anionic groups, such as carboxylate, thioglycolate, sulphosuccinate, thiosulphate, phosphate, and sulphate groups (so-called “organomodified” silicones).

As used herein, the term “polysiloxane macromer” means, in conformity with what is generally accepted, any monomer comprising, in its structure, a polymer chain of the polysiloxane type.

The polymers comprising a non-silicone organic backbone grafted by monomers comprising a polysiloxane used according to the present disclosure can comprise an organic main chain formed from organic monomers not comprising silicone, to which is grafted, within the said chain and optionally at at least one of its ends, at least one polysiloxane macromer.

The non-silicone organic monomers constituting the main chain of the grafted silicone polymer can be chosen, for example, from monomers comprising ethylenic unsaturation polymerizable by the radical route, monomers polymerizable by polycondensation, such as those forming polyamides, polyesters, and polyurethanes, and ring-opening monomers, such as oxazoline monomers and caprolactone monomers.

The polymers comprising a non-silicone organic backbone grafted by monomers comprising a polysiloxane may be obtained by any means known to a person skilled in the art, for example, by reaction between (i) a starting polysiloxane macromer correctly functionalized on the polysiloxane chain and (ii) at least one non-silicone organic compound correctly functionalized by a functional group which is capable of reacting with the at least one functional group carried by the silicone with the formation of a covalent bond; a classic example of such a reaction is, for instance, the radical reaction between a vinyl group carried on one of the ends of the silicone and a double bond of a monomer comprising ethylenic unsaturation of the main chain.

The polymers comprising a non-silicone organic backbone grafted by monomers comprising a polysiloxane in accordance with the present disclosure may be chosen, for example, from those disclosed in U.S. Pat. Nos. 4,693,935, 4,728,571, and 4,972,037, European Patent Application Nos. 0 412 704, 0 412 707, and 0 640 105, and International Patent Application No. WO 95/00578. They may be chosen from copolymers obtained by radical polymerization from monomers comprising ethylenic unsaturation and from silicone macromers having an end vinyl group and copolymers obtained by reaction of a polyolefin comprising functionalized groups and of a polysiloxane macromer having an end functional group which is reactive with the functionalized groups.

A non-limiting example of a family of grafted silicone polymers which is suitable for implementing the present disclosure includes the grafted silicone copolymers comprising:

a) from 0 to 98% by weight of at least one lipophilic monomer (A) of low lipophilic polarity comprising ethylenic unsaturation which is polymerizable by the radical route;

b) from 1 to 98% by weight of at least one polar hydrophilic monomer (B) comprising ethylenic unsaturation which is copolymerizable with the at least one monomer of the (A) type; and

c) from 0.01 to 50% by weight of at least one polysiloxane macromer (C) of formula (II):

X(Y)ₙSi(R)₃₋ₘZₘ (II)

wherein:

X is a vinyl group which is copolymerizable with the monomers (A) and (B);

Y is a group with divalent bonding;

R is chosen from hydrogen, C₁-C₆ alkyl groups, C₁-C₆ alkoxy groups, and C₆-C₁₂ aryl groups;

Z is a monovalent polysiloxane unit having a number-average molecular weight of at least 500;

n is equal to 0 or 1; and

m is an integer ranging from 1 to 3;

the percentages being calculated with respect to the total weight of the monomers (A), (B), and (C).

These polymers, and processes for the preparation thereof, are disclosed, for example, in U.S. Pat. Nos. 4,963,935, 4,728,571, and 4,972,037 and European Patent Application Nos. 0 412 704, 0 412 707, and 0 640 105. They can have a number-average molecular weight ranging from 10,000 to 2,000,000 and a glass transition temperature T_{g} or a crystalline melting point Tₘ of at least −20° C.

Examples of lipophilic monomers (A) include, but are not limited to, esters of acrylic or methacrylic acid with C₁-C₁₈ alcohols; styrene; polystyrene macromers; vinyl acetate; vinyl propionate; α-methylstyrene; tert-butylstyrene; butadiene; cyclohexadiene; ethylene; propylene; vinyltoluene; esters of acrylic or methacrylic acid with 1,1-dihydroperfluoroalkanol or with its homologues; esters of acrylic or methacrylic acid with ω-hydridofluoroalkanol; esters of acrylic or methacrylic acid with fluoroalkylsulphonamidoalcohol; esters of acrylic or methacrylic acid with fluoroalkyl alcohol; esters of acrylic or methacrylic acid with alcohol fluoroether; and mixtures thereof.

In at least one embodiment, the monomers (A) may be chosen from n-butyl methacrylate, isobutyl methacrylate, tert-butyl acrylate, tert-butyl methacrylate, 2-ethylhexyl methacrylate, methyl methacrylate, 2-(N-methylperfluorooctanesulphonamido)ethyl acrylate, 2-(N-butylperfluorooctanesulphonamido)ethyl acrylate, and mixtures thereof.

Examples of polar monomers (B) include, but are not limited to, acrylic acid, methacrylic acid, N,N-dimethylacrylamide, dimethylaminoethyl methacrylate, quaternized dimethylaminoethyl methacrylate, (meth)acrylamide, N-t-butylacrylamide, maleic acid, maleic anhydride and their hemi-esters, hydroxyalkyl (meth)acrylates, diallyldimethylammonium chloride, vinylpyrrolidone, vinyl ethers, maleimides, vinylpyridine, vinylimidazole, polar heterocyclic vinyl compounds, styrenesulphonate, allyl alcohol, vinyl alcohol, vinylcaprolactam, and mixtures thereof. In one embodiment, the monomers (B) may be chosen from acrylic acid, N,N-dimethylacrylamide, dimethylaminoethyl methacrylate, quaternized dimethylaminoethyl methacrylate, vinylpyrrolidone, and mixtures thereof.

In another embodiment, as polar monomers (B), the anionic grafted silicone polymers used according to the present disclosure comprise at least one anionic monomer.

The polysiloxane macromers (C) of formula (I) may be chosen, for example, from those of formula (III):

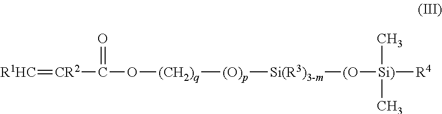

wherein:

R¹ is chosen from hydrogen and —COOH, and in at least one embodiment, hydrogen;

R² is chosen from hydrogen, methyl, and —CH₂COOH, and in at least one embodiment, methyl;

R³ is chosen from C₁-C₆ alkyl groups, C₁-C₆ alkoxy groups, C₁-C₆ alkylamino groups, C₆-C₁₂ aryl groups, and hydroxyl groups, and in at least one embodiment, methyl groups;

R⁴ is chosen from C₁-C₆ alkyl groups, C₁-C₆ alkoxy groups, C₁-C₆ alkylamino groups, C₆-C₁₂ aryl groups, and hydroxyl groups, and in at least one embodiment, methyl groups;

q is an integer ranging from 2 to 6, and in at least one embodiment, is equal to 3;

p is equal to 0 or 1;

r is an integer ranging from 5 to 700; and

m is an integer ranging from 1 to 3, and in at least one embodiment, is equal to 1.

In another embodiment, the polysiloxane macromers (C) may be chosen from those of formula (IV):

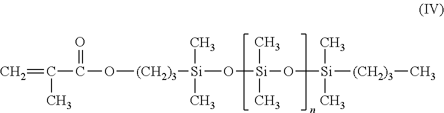

wherein n is an integer ranging from 5 to 700.

According to another embodiment of the present disclosure, use may be made of a copolymer capable of being obtained by radical polymerization from a mixture of monomers comprising:

a) 60% by weight of tert-butyl acrylate;

b) 20% by weight of acrylic acid;

c) 20% by weight of silicone macromer of formula (V):

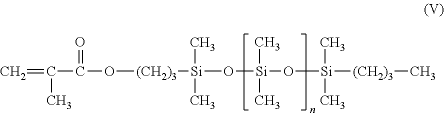

wherein n is an integer ranging from 5 to 700; the percentages by weight being calculated with respect to the total weight of the monomers.

Another family of silicone polymers which is suitable for use in accordance with the present disclosure comprises grafted silicone copolymers capable of being obtained by reactive extrusion of a polysiloxane macromer having an end reactive functional group with a polymer of the polyolefin type comprising reactive groups capable of reacting with the end functional group of the polysiloxane macromer to form a covalent bond which makes possible the grafting of the silicone to the main chain of the polyolefin.

These polymers and their process of preparation are disclosed, for example, in International Patent Application Publication No. WO 95/00578.

The reactive polyolefins may be chosen, for example, from polyethylenes and polymers of monomers derived from ethylene, such as propylene, styrene, alkylstyrenes, butylene, butadiene, (meth)acrylates, vinyl esters, and equivalents, comprising reactive functional groups capable of reacting with the end functional group of the polysiloxane macromer. They may be chosen, in at least one embodiment, from copolymers of ethylene or of ethylene derivatives and of monomers chosen from those comprising a carboxyl functional group, such as (meth)acrylic acid; those comprising an acid anhydride functional group, such as maleic anhydride; those comprising an acid chloride functional group, such as (meth)acryloyl chloride; those comprising an ester functional group, such as (meth)acrylates; and those comprising an isocyanate functional group.

The silicone macromers may be chosen, for example, from polysiloxanes comprising a functionalized group, at the end of the polysiloxane chain or close to the end of the said chain, chosen from alcohols, thiols, epoxys, and primary and secondary amines, and in at least one embodiment, those corresponding to formula (VI):

T-(CH₂)ₛ—Si—[(OSiR⁵R⁶)ₜR⁷]_{y} (VI)

wherein:

T is chosen from NH₂, NHR′, epoxy functional groups, OH, and SH;

R⁵, R⁶, R⁷, and R′, which may be identical or different, are chosen from C₁-C₆ alkyl groups, phenyl groups, benzyl groups, C₆-C₁₂ alkylphenyl groups, and hydrogen;

s is a number ranging from 2 to 100;

t is a number ranging from 0 to 1000; and

y is a number ranging from 1 to 3.

These silicone macromers may have a number-average molecular weight ranging from 5,000 to 300,000, for instance, from 8,000 to 200,000, or from 9,000 to 40,000.

According to at least one embodiment of the present disclosure, the at least one grafted silicone polymer comprising a polysiloxane backbone grafted by non-silicone organic monomers comprise a main silicone (or polysiloxane (≡Si—O—)ₙ) chain to which is grafted, within the said chain and optionally at at least one of its ends, at least one organic group not comprising silicone.

The polymers comprising a polysiloxane backbone grafted by non-silicone organic monomers according to the present disclosure may be chosen from existing commercial products, or alternatively, may be obtained according to methods known to a person skilled in the art, for example, by reaction between (i) a starting silicone correctly functionalized on at least one of its silicon atoms and (ii) a non-silicone organic compound correctly functionalized by a functional group which is capable of reacting with the at least one functional group carried by the silicone with the formation of a covalent bond; such as the hydrosilylation reaction between ≡Si—H groups and CH₂═CH— vinyl groups and the reaction between —SH thio-functional groups and these same vinyl groups.

Examples of polymers comprising a polysiloxane backbone grafted by non-silicone organic monomers suitable for use in accordance with the present disclosure, as well as their method of preparation, are disclosed, for example, in European Patent Application No. 0 582 152 and International Patent Application Publication Nos. WO 93/23009 and WO 95/03776, the teachings of which are incorporated herein by reference in their entireties.

According to another embodiment of the present disclosure, the silicone polymer comprising a polysiloxane backbone grafted by non-silicone organic monomers use polymers resulting from the radical copolymerization between at least one anionic non-silicone organic monomer exhibiting an ethylenic unsaturation and/or one hydrophobic non-silicone organic monomer exhibiting an ethylenic unsaturation and a silicone exhibiting, in its chain, at least one functional group capable of reacting with the ethylenic unsaturations of the non-silicone monomers with the formation of a covalent bond, for instance, thiofunctional groups.

According to at least one embodiment of the present disclosure, the anionic monomers comprising ethylenic unsaturation may be chosen from unsaturated, linear or branched, carboxylic acids, optionally partially or completely neutralized in the form of a salt, and mixtures thereof, it being possible for the unsaturated carboxylic acids to be chosen from acrylic acid, methacrylic acid, maleic acid, maleic anhydride, itaconic acid, fumaric acid, and crotonic acid. Suitable salts include, for instance, alkali metal, alkaline earth metal, and ammonium salts. It should be noted that, likewise, in the final grafted silicone polymer, the organic group of anionic nature of the result of the radical (homo)polymerization of at least one anionic monomer of unsaturated carboxylic acid type can, after reaction, be post-neutralized with a base (e.g., sodium hydroxide, ammonia, and the like) in order to convert it into a salt form.

According to one embodiment of the present disclosure, the hydrophobic monomers comprising ethylenic unsaturation may be chosen from alkanol acrylic acid esters, alkanol methacrylic acid esters, and mixtures thereof. The alkanols may be chosen, for example, from C₁-C₁₈ alkanols, such as C₁-C₁₂ alkanols. In at least one embodiment, the monomers may be chosen from isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, stearyl (meth)acrylate, and mixtures thereof.

A non-limiting example of a family of silicone polymers comprising a polysiloxane backbone grafted by non-silicone organic monomers which is suitable for use in accordance with the present disclosure includes silicone polymers comprising, in their structure, at least one unit chosen from those of formula (VII):

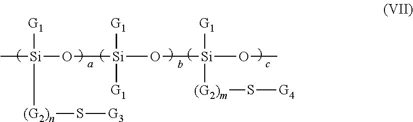

wherein:

the G₁ radicals, which may be identical or different, are chosen from hydrogen, C₁-C₁₀ alkyl radicals, and phenyl radicals;

the G₂ radicals, which may be identical or different, are chosen from C₁-C₁₀ alkylene groups;

G₃ is a polymer residue resulting from the (homo)polymerization of at least one anionic monomer comprising ethylenic unsaturation;

G₄ is a polymer residue resulting from the (homo)polymerization of at least one hydrophobic monomer comprising ethylenic unsaturation;

m and n, which may be identical or different, are equal to 0 or 1;

a is an integer ranging from 0 to 50;

b is an integer ranging from 10 to 350; and

c is an integer ranging from 0 to 50, with the proviso that one of the parameters chosen from a and c is other than 0.

According to one embodiment, the above unit of formula (VII) may exhibit at least one or all of the following characteristics:

the G₁ radicals are chosen from alkyl radicals, and in at least one embodiment, methyl radicals;

n is not zero and the G₂ radicals are chosen from divalent C₁-C₃ radicals, and in at least one embodiment, propylene radicals;

G₃ is a polymer radical resulting from the (homo)polymerization of at least one monomer of the carboxylic acid comprising ethylenic unsaturation type, and in at least one embodiment, acrylic acid and/or methacrylic acid;

G₄ is a polymer radical resulting from the (homo)polymerization of at least one monomer of the (C₁-C₁₀)alkyl (meth)acrylate type, and in at least one embodiment, isobutyl (meth)acrylate or methyl (meth)acrylate.

Examples of grafted silicone polymers corresponding to the formula (IV) include, but are not limited to, polydimethylsiloxanes (PDMS) to which are grafted, via a connecting link of thiopropylene type, mixed polymer units of the poly((meth)acrylic acid) type and of the poly(methyl (meth)acrylate) type.

The number-average molecular weight of the silicone polymers comprising a polysiloxane backbone grafted by non-silicone organic monomers of the present disclosure may range from 10,000 to 1,000,000, for example, from 10,000 to 100,000.

A non-limiting example of a grafted silicone polymer which can be used according to the present disclosure is the product sold by 3M under the reference VS80.

H) Anionic polyurethanes.

In at least one embodiment, the polyurethanes used according to the present disclosure may comprise a base repeat unit of formula (VIII):

—X′—B—X′—CO—NH—R—NH—CO— (VIII)

wherein:
X′, which may be identical or different, is chosen from O and NH,
B is a substituted or unsubstituted divalent hydrocarbon radical, and
R is a divalent radical chosen from branched or unbranched C₆-C₂₀ aromatic alkylene radicals; C₁ to C₂₀, for example, C₁-C₆, aliphatic alkylene radicals; and C₁ to C₂₀, for instance, C₁-C₆, cycloaliphatic alkylene radicals, these radicals being unsubstituted or substituted by at least one entity chosen from halogen, C₁-C₄ alkoxy radicals, and C₆-C₃₀ aryl radicals, for instance, phenyl groups.

In at least one embodiment, the B radical is a divalent C₁-C₃₀, for example, C₂-C₁₀, radical and carries a group comprising at least one functional group chosen from carboxyl functional groups and sulpho functional groups, the carboxyl and/or sulpho functional groups being in the free form or in the form partially or completely neutralized by an inorganic or organic base, such as alkali metal hydroxides, alkaline earth metal hydroxides, ammonia, alkylamines, alkanolamines, and organic amino acids. In another embodiment, B is the divalent radical resulting from dimethylolpropionic acid.

In yet another embodiment, the R radical is chosen from radicals of the following formulae:

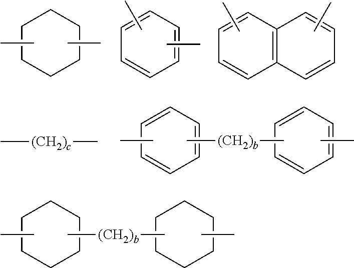

wherein b is an integer ranging from 0 to 3 and c is an integer ranging from 1 to 20, for example, from 2 to 12.

In a further embodiment, the R radical is chosen from hexamethylene, 4,4′-biphenylenemethane, 2,4- and/or 2,6-tolylene, 1,5-naphthylene, p-phenylene, and methylene-4,4-biscyclohexyl radicals and the divalent radical derived from isophorone.

Suitable fixing polyurethanes can comprise, for example, silicone grafts and silicones comprising hydrocarbon grafts.

A non-limiting example of a suitable polyurethane is one comprising, in addition, at least one polysiloxane sequence and a base repeat unit corresponding, for example, to formula (IX):

—X′—P—X′—CO—NH—R—NH—CO— (IX)

wherein:
P is a polysiloxane segment,
X′, which may be identical or different, is chosen from O and NH, and
R is a divalent radical chosen from branched or unbranched C₆-C₂₀ aromatic alkylene radicals, C₁-C₂₀, for example, C₁-C₆, aliphatic alkylene radicals, and C₁-C₂₀, for instance, C₁-C₆, cycloaliphatic alkylene radicals, these radicals being unsubstituted or substituted by at least one entity chosen from halogen, C₁-C₄ alkoxy radicals, and C₆-C₃₀ aryl radicals, for example, phenyl groups.

According to one embodiment, the R radical may be chosen from radicals of the following formulae:

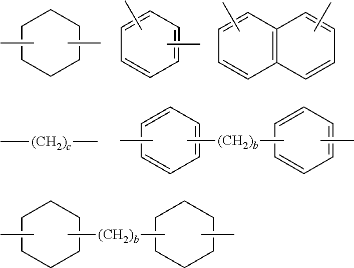

wherein b is an integer ranging from 0 to 3 and c is an integer ranging from 1 to 20, for example, from 2 to 12.

For example, the R radical may be chosen from hexamethylene, 4,4′-biphenylenemethane, 2,4- and/or 2,6-tolylene, 1,5-naphthylene, p-phenylene, and methylene-4,4-biscyclohexyl radicals and the divalent radical derived from isophorone.

According to at least one embodiment, the polysiloxane segment P corresponds to formula (X):

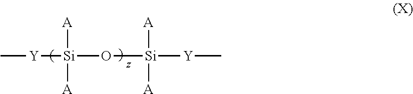

wherein:
the A groups, which may be identical or different, are chosen from monovalent C₁ to C₂₀ hydrocarbon groups substantially devoid of ethylenic unsaturation and aromatic groups,
Y is a divalent hydrocarbon group, and
Z is an integer chosen such that the average molecular weight of the polysiloxane segment ranges from 300 to 10,000.

In one embodiment, the divalent group Y is chosen from alkylene groups of formula —(CH₂)ₐ— wherein a is an integer ranging from 1 to 10.

The A groups may be chosen, for example, from C₁-C₁₈ alkyl groups, such as methyl, ethyl, propyl, isopropyl, butyl, pentyl, hexyl, octyl, decyl, dodecyl, and octadecyl groups; cycloalkyl groups, such as cyclohexyl groups; aryl groups, such as phenyl and naphthyl groups; arylalkyl groups, such as benzyl and phenylethyl groups, tolyl groups, and xylyl groups.

Examples of fixing polyurethanes include, but are not limited to the dimethylolpropionic acid/isophorone diisocyanate/neopentyl glycol/polyesterdiols copolymer (also known under the name of polyurethane-1, INCI name) sold under the name Luviset® PUR by BASF and the dimethylolpropionic acid/isophorone diisocyanate/neopentyl glycol/polyesterdiols/silicone diamine copolymer (also known under the name of polyurethane-6, INCI name) sold under the name Luviset® Si PUR A by BASF.

A non-limiting example of a suitable anionic polyurethane is Avalure UR 450.

Use may also be made of polymers comprising sulphoisophthalate groups, such as the AQ55 and AQ48 polymers sold by Eastman.

According to one embodiment of the present disclosure, the anionic polymers may be chosen from acrylic acid copolymers, such as the acrylic acid/ethyl acrylate/N-tert-butylacrylamide terpolymer sold under the name Ultrahold Strong® by BASF, methacrylic acid/ethyl acrylate copolymers, for example, those in aqueous dispersion, such as Luviflex Soft and Luvimer MAE, sold by BASF, copolymers derived from crotonic acid, such as the vinyl acetate/vinyl tert-butylbenzoate/crotonic acid terpolymers and the crotonic acid/vinyl acetate/vinylneododecanoate terpolymers sold under the name Resin 28-29-30 by National Starch, polymers derived from maleic, fumaric or itaconic acids or anhydrides with vinyl esters, vinyl ethers, vinyl halides, phenylvinyl derivatives, acrylic acid, and its esters, such as the monoesterified methyl vinyl ether/maleic anhydride copolymer sold under the name GANTREZ® ES 425 by ISP, Luviset Si PUR, Mexomere PW, elastomeric or nonelastomeric anionic polyurethanes, polymers comprising sulphoisophthalate groups, anionic grafted silicone polymers, and Amerhold DR 25 and VS 80.

The film-forming cationic fixing polymers which are suitable for use according to the present disclosure can be chosen, for example, from polymers comprising primary, secondary, tertiary, and/or quaternary amine groups forming part of the polymer chain or directly connected to the latter and having a molecular weight ranging from 500 to 5,000,000, for example, from 1,000 to 3,000,000.

In at least one embodiment, the film-forming cationic fixing polymers may be chosen from:

(1) Homopolymers or copolymers derived from acrylic or methacrylic esters or amides comprising at least one unit chosen from units of formulae (A)-(C):

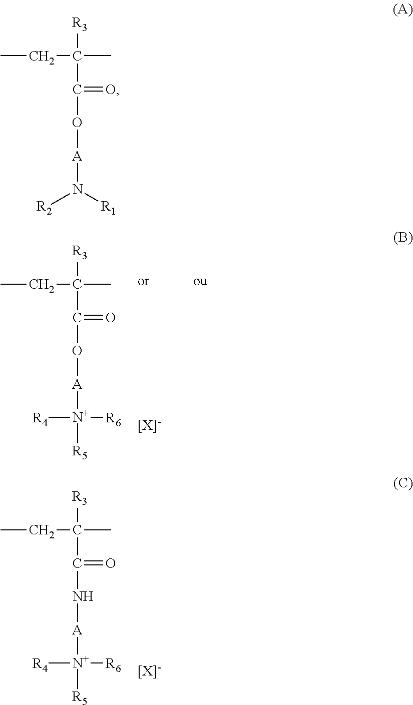

wherein:

R₃ is chosen from hydrogen and CH₃ radicals;

A is chosen from linear and branched alkyl groups comprising from 1 to 6 carbon atoms and hydroxyalkyl groups comprising from 1 to 4 carbon atoms;

R₄, R₅, and R₆, which may be identical or different, are chosen from alkyl groups comprising from 1 to 18 carbon atoms and benzyl radicals;

R₁ and R₂, which may be identical or different, are chosen from hydrogen and alkyl groups comprising from 1 to 6 carbon atoms; and

X is chosen from methyl sulphate anions and halides, such as chloride and bromide.

The copolymers of family (1) additionally comprise at least one unit deriving from comonomers chosen, for example, from acrylamides, methacrylamides, diacetone acrylamides, acrylamides and methacrylamides substituted on the nitrogen by lower (C₁-C₄) alkyl groups, groups derived from acrylic or methacrylic acids or esters thereof, vinyllactams, such as vinylpyrrolidone and vinylcaprolactam, and vinyl esters.

Non-limiting examples of copolymers of family (1) include:
copolymers of acrylamide and of dimethylaminoethyl methacrylate quaternized with dimethyl sulphate or with a methyl halide, such as that sold under the name Hercofloc® by Hercules,
copolymers of acrylamide and of methacryloyloxyethyltrimethylammonium chloride, disclosed, for example, in European Patent Application No. 0 080 976 and sold under the name Bina Quat P 100 by Ciba-Geigy,
copolymer of acrylamide and of methacryloyloxyethyltrimethylammonium methyl sulphate, such as that sold under the name Reten by Hercules,
vinylpyrrolidone/dialkylaminoalkyl acrylate and methacrylate copolymers, which may or may not be quaternized, such as the products sold under the name “Gafquat®” by ISP, for example “Gafquat® 734” and “Gafquat® 755”, and the products named “Copolymer® 845, 958, and 937”. These polymers are described, for example, in French Patent Nos. 2 077 143 and 2 393 573,
polymers comprising a fatty chain and comprising a vinylpyrrolidone unit, such as the products sold under the names Styleze W20 and Styleze W10 by ISP,
dimethylaminoethyl methacrylate/vinylcaprolactam/vinylpyrrolidone terpolymers, such as the product sold under the name Gaffix VC 713 by ISP, and
quaternized vinylpyrrolidone/dimethylaminopropylmethacrylamide copolymers, such as the products sold under the name “Gafquat® HS 100” by ISP.

(2) Cationic guar gums, for instance, those comprising quaternary ammonium, such as those disclosed in U.S. Pat. Nos. 3,589,578 and 4,031,307, such as guar gums comprising trialkylammonium cationic groups. Such products are sold, for example, under the trade names Jaguar C13 S, Jaguar C15, and Jaguar C17 by Meyhall.

(3) Quaternary copolymers of vinylpyrrolidone and of vinylimidazole.

(4) Chitosans and their salts; the salts being chosen, for example, from chitosan acetate, lactate, glutamate, gluconate, and pyrrolidonecarboxylate.

Examples of suitable commercial products include, but are not limited to, chitosan having a degree of deacetylation of 90.5% by weight sold under the name Kytan Brut Standard by Aber Technologies and the chitosan pyrrolidonecarboxylate sold under the name Kytamer® PC by Amerchol.

(5) Cationic cellulose derivatives, such as the copolymers of cellulose or of cellulose derivatives grafted with a water-soluble monomer comprising a quaternary ammonium and disclosed, for instance, in U.S. Pat. No. 4,131,576, such as hydroxyalkylcelluloses, for example hydroxymethyl-, hydroxyethyl-, and hydroxypropylcelluloses, grafted, for example, with an ammonium salt chosen from methacryloyloxyethyltrimethylammonium, methacrylamidopropyltrimethylammonium, and dimethyldiallylammonium salts.

Commercial products corresponding to this definition include, but are not limited to the products sold under the names “Celquat L 200” and “Celquat H 100” by National Starch.

Amphoteric fixing polymers which can be used in accordance with the present disclosure can be chosen, for example, from polymers comprising B and C units distributed randomly in the polymer chain, wherein B is a unit deriving from a monomer comprising at least one basic nitrogen atom and C is a unit deriving from an acidic monomer comprising at least one group chosen from carboxyl and sulpho groups, or else B and C may be chosen from groups deriving from monomers chosen from zwitterionic carboxybetaine monomers and zwitterionic sulphobetaine monomers; B and C may also form a cationic polymer chain comprising at least one group chosen from primary, secondary, tertiary, and quaternary amine groups, in which at least one of the amine groups carries a carboxyl or sulpho group connected via a hydrocarbon group, or else B and C may form part of a chain of a polymer comprising a dicarboxyethylene unit, one of the carboxyl groups of which having been reacted with a polyamine comprising at least one group chosen from primary and secondary amine groups.

In at least one embodiment, the amphoteric polymers may be chosen from:

1) polymers resulting from the copolymerization of a monomer derived from a vinyl compound carrying a carboxyl group, such as acrylic acid, methacrylic acid, maleic acid, and α-chloracrylic acid, and of a basic monomer derived from a substituted vinyl compound comprising at least one basic atom, such as dialkylaminoalkyl methacrylate and acrylate and dialkylaminoalkylmethacrylamide and -acrylamide. Such compounds are disclosed, for instance, in U.S. Pat. No. 3,836,537.

The vinyl compound may also be chosen from dialkyldiallylammonium salts, such as diethyldiallylammonium chloride.

2) Polymers comprising units deriving:

a) from at least one monomer chosen from acrylamides and methacrylamides substituted on the nitrogen atom by an alkyl group,

b) from at least one acidic comonomer comprising at least one reactive carboxyl group, and

c) from at least one basic comonomer, such as esters comprising primary, secondary, tertiary, and quaternary amine substituents of acrylic and methacrylic acids and the quaternization product of dimethylaminoethyl methacrylate with dimethyl or diethyl sulphate.

N-substituted acrylamides or methacrylamides suitable for use in accordance with the present disclosure include compounds in which the alkyl groups comprise from 2 to 12 carbon atoms, for example, N-ethylacrylamide, N-tert-butylacrylamide, N-tert-octylacrylamide, N-octylacrylamide, N-decylacrylamide, N-dodecylacrylamide, and the corresponding methacrylamides.

The acidic comonomers may be chosen, for example, from acrylic, methacrylic, crotonic, itaconic, maleic, and fumaric acids and alkyl monoesters having 1 to 4 carbon atoms of maleic or fumaric acids or anhydrides. The basic comonomers may be chosen, for instance, from aminoethyl, butylaminoethyl, N,N′-dimethylaminoethyl, and N-tert-butylaminoethyl methacrylates. The copolymers for which the CTFA name (4th Ed., 1991) is octylacrylamide/acrylates/butylaminoethyl methacrylate copolymer, such as the products sold under the name Amphomer® and Lovocryl® 47 by National Starch may also be used.

(3) Partially or completely alkylated and crosslinked polyaminoamides deriving from polyaminoamides of formula (XI):

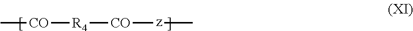

wherein R₄ is a divalent group derived from a saturated dicarboxylic acid, from an aliphatic mono- or dicarboxylic acid comprising an ethylenic double bond, from an ester of a lower alkanol comprising from 1 to 6 carbon atoms of these acids, or from a group deriving from the addition of any one of the above acids with a bisprimary or bissecondary amine, and Z is a group derivative from a bisprimary, mono- or bissecondary polyalkylenepolyamine and in at least one embodiment, comprises:

a) in an amount ranging from 60 to 100 mol %, groups chosen from those of formula (XII):

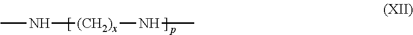

wherein x=2 and p=2 or 3, or else x=3 and p=2

this group deriving from an amine chosen from diethylenetriamine, triethylene-tetraamine, and dipropylenetriamine;

b) in an amount ranging from 0 to 40 mol %, a group chosen from those of formula (XII), wherein x=2 and p=1 and which derives from ethylenediamine, and groups deriving from piperazine:

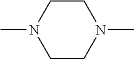

c) in an amount ranging from 0 to 20 mol %, the group —NH—(CH₂)₆—NH— deriving from hexamethylenediamine, these polyaminoamides being crosslinked by addition of a bifunctional crosslinking agent chosen from epihalohydrins, diepoxides, dianhydrides, and bisunsaturated derivatives, by means of 0.025 to 0.35 mol of crosslinking agent per amine group of the polyaminoamide, and alkylated by reaction with an entity chosen from acrylic acid, chloroacetic acid, alkanesultones, and salts thereof.

The saturated carboxylic acids may be chosen, for example, from acids comprising from 6 to 10 carbon atoms, such as adipic acid, 2,2,4-trimethyladipic acid, 2,4,4-trimethyladipic acid, terephthalic acids, and acids comprising an ethylenic double bond, for example, acrylic, methacrylic, and itaconic acids. The alkanesultones used in the alkylation may be chosen, for instance, from propanesultone and butanesultone and the salts of the alkylating agents may be chosen, for example, from sodium and potassium salts.

(4) Polymers comprising zwitterionic units of formula (XIII):

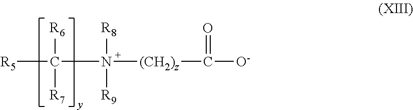

wherein:

R₅ is a polymerizable unsaturated group, such as acrylate, methacrylate, acrylamide, and methacrylamide groups,

y and z, which may be identical or different, are integers ranging from 1 to 3,

R₆ and R₇, which may be identical or different, are chosen from hydrogen, methyl groups, ethyl groups, and propyl groups, and

R₈ and R₉, which may be identical or different, are chosen from hydrogen and alkyl groups such that the sum of the carbon atoms in R₈ and R₉ does not exceed 10.

In at least one embodiment, the polymers comprising such units may also comprise units derived from non-zwitterionic monomers, such as dimethyl- or diethylaminoethyl acrylate or methacrylate, alkyl acrylates or methacrylates, acrylamides or methacrylamides, and vinyl acetate.

(5) Polymers derived from chitosan comprising monomer units of formulae (XIV)-(XVI):

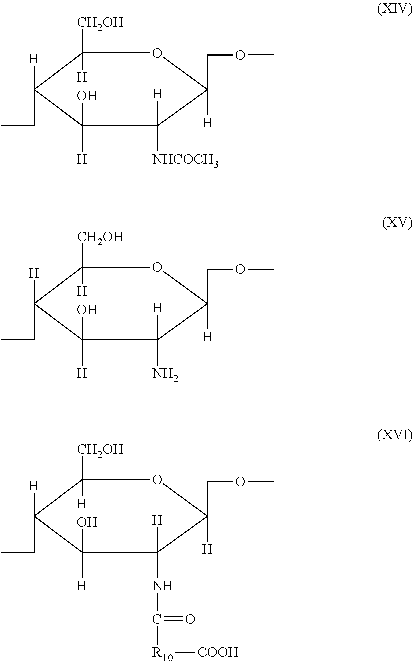

the unit (XIV) being present in an amount ranging from 0 to 30%, the unit (XV) in an amount ranging from 5 to 50%, and the unit (XVI) in an amount ranging from 30 to 90%, it being understood that, in this unit (XVI), R₁₀ is a group of formula (XVII):

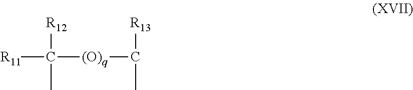

wherein, if q=0, R₁₁, R₁₂ and R₁₃, which may be identical or different, are chosen from hydrogen, methyl residues, hydroxyl residues, acetoxy residues, amino residues, monoalkylamine residues, and dialkylamine residues, optionally interrupted by at least one nitrogen atom and/or optionally substituted by at least one group chosen from amine, hydroxyl, carboxyl, alkylthio, and sulpho groups, and alkylthio residues in which the alkyl group carries an amino residue, at least one of the R₁₁, R₁₂ and R₁₃ groups being, in this case, a hydrogen atom;

or, if q=1, R₁₁, R₁₂, and R₁₃ are each hydrogen, and the acid and base addition salts thereof.

(6) Polymers derived from the N-carboxyalkylation of chitosan.

(7) Polymers of units of formula (XVIII), which are, for example, disclosed in French Patent 1 400 366:

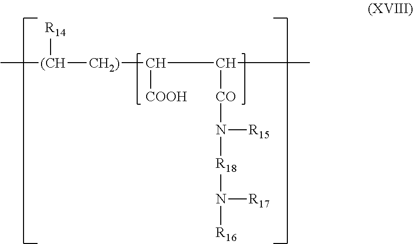

wherein:

R₁₄ is chosen from hydrogen, CH₃O, CH₃CH₂O, and phenyl groups,

R₁₅ is chosen from hydrogen and lower alkyl groups, such as methyl and ethyl,

R₁₆ is chosen from hydrogen and lower alkyl groups, such as methyl and ethyl, and

R₁₇ is chosen from lower alkyl groups, such as methyl and ethyl, and groups corresponding to the formula: —R₁₈—N(R₁₆)₂, wherein R₁₈ is chosen from —CH₂—CH₂—, —CH₂—CH₂—CH₂—, and —CH₂—CH(CH₃)— groups, and R₁₆ is defined above,

and the higher homologues of these groups comprising up to 6 carbon atoms.

(8) Amphoteric polymers of the -D-X-D-X— type chosen from:

a) polymers obtained by reaction of chloroacetic acid or sodium chloroacetate with compounds comprising at least one unit of formula (XIX):

-D-X-D-X-D- (XIX)

wherein D is a group

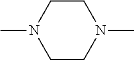

and X is chosen from the symbols E and E′, wherein E and E′, which may be identical or different, are chosen from bivalent groups which are straight- or branched-chain alkylene groups comprising up to 7 carbon atoms in the main chain which can be unsubstituted or substituted by hydroxyl groups and which can additionally comprise oxygen, nitrogen or sulphur atoms or 1 to 3 aromatic and/or heterocyclic rings; the oxygen, nitrogen and sulphur atoms being present in the form of ether, thioether, sulphoxide, sulphone, sulphonium, alkylamine, alkenylamine, hydroxyl, benzylamine, amine oxide, quaternary ammonium, amide, imide, alcohol, ester, and/or urethane groups.

b) polymers of formula:

-D-X-D-X— (XX)

wherein D is a group

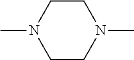

and X is chosen from the symbols E and E′, and at least once E′, wherein E is as defined above and E′ is a bivalent group which is a straight- or branched-chain alkylene group comprising up to 7 carbon atoms in the main chain which can be unsubstituted or substituted by at least one hydroxyl group and which comprises at least one nitrogen atom, the nitrogen atom being substituted by an alkyl chain optionally interrupted by an oxygen atom and comprising at least one group chosen from carboxyl functional groups and hydroxyl functional groups and betainized by reaction with chloroacetic acid or sodium chloroacetate.

(9) (C₁-C₅)Alkyl vinyl ether/maleic anhydride copolymers partially modified by semiamidation with an N,N-dialkylaminoalkylamine, such as N,N-dimethylamino-propylamine, or by semiesterification with an N,N-dialkanolamine. These copolymers may also comprise other vinyl comonomers, such as vinylcaprolactam.

According to one embodiment of the present disclosure, the fixing amphoteric polymers may be chosen from branched block copolymers comprising:

(a) nonionic units derived from at least one monomer chosen from C₁-C₂₀ alkyl (meth)acrylates, N-mono(C₂-C₁₂ alkyl)(meth)acrylamides, and N,N-di(C₂-C₁₂ alkyl) (meth)acrylamides,

(b) anionic units derived from at least one monomer chosen from acrylic acid and methacrylic acid, and
(c) polyfunctional units derived from at least one monomer comprising at least two polymerizable unsaturated functional groups,

and, in at least one embodiment, having a structure comprising hydrophobic blocks to which are attached, via polyfunctional units (c), several more hydrophilic blocks.

According to another embodiment, the amphoteric polymers may exhibit at least two glass transition temperatures (Tg), at least one of which is greater than 20° C. and the other of which is less than 20° C.

The amphoteric polymers may be chosen, for example, from polymers comprising units deriving:

a) from at least one monomer chosen from acrylamides and methacrylamides substituted on the nitrogen by an alkyl group,

b) from at least one acidic comonomer comprising at least one reactive carboxyl group, and

c) from at least one basic comonomer, such as esters comprising primary, secondary, tertiary, and quaternary amine substituents of acrylic and methacrylic acids and the quaternization product of dimethylaminoethyl methacrylate with dimethyl or diethyl sulphate.

Non-limiting examples of suitable commercial product are the polymers sold under the name Amphomer by National Starch.

Generally, the at least one fixing polymer is present in the composition in an amount ranging from 0.1 to 20%, for example, from 1 to 15%, by weight of the total weight of the composition.

In at least one embodiment, the composition according to the present disclosure further comprises at least one thickening agent chosen from ionic and nonionic thickening agents, also referred to as “rheology-adjusting agents.”

The at least one rheology-adjusting agent can be chosen, for example, from fatty acid amides (e.g., coconut diethanol- or monoethanolamide, oxyethylenated alkyl ether carboxylic acid monoethanolamide). According to one embodiment, the at least one rheology-adjusting agent is polymeric. These polymeric agents can be chosen, for example, from cellulose thickeners (e.g., hydroxyethylcellulose, hydroxypropylcellulose, carboxymethylcellulose), guar gum and its derivatives (e.g., hydroxypropyl guar), gums of microbial origin (e.g., xanthan gum, scleroglucan gum), crosslinked homopolymers or copolymers of acrylic acid or of acrylamidopropanesulphonic acid, and associative thickening polymers, such as those described below. In another embodiment, the polymeric agent may be chosen from crosslinked homopolymers or copolymers of acrylic acid or of acrylamidopropanesulphonic acid, for example, from crosslinked homopolymers or copolymers of acrylamidopropanesulphonic acid.

The at least one thickening agent may be present in the composition in an amount ranging from 0.01 to 10%, for example, from 0.1 to 5%, or from 0.3 to 3%, by weight, with respect to the total weight of the composition.

Associative polymers are water-soluble polymers capable, in an aqueous medium, of reversibly associating with one another or with other molecules. Their chemical structure comprises hydrophilic regions and hydrophobic regions characterized by at least one fatty chain.

The associative polymers can be chosen from anionic, cationic, amphoteric, and nonionic polymers.

Examples of anionic associative polymers include, but are not limited to:

(I) those comprising at least one hydrophilic unit and at least one allyl ether unit comprising a fatty chain, for instance, those for which the hydrophilic unit comprises at least one ethylenic unsaturated anionic monomer, such as vinyl carboxylic acids, for instance, acrylic acid, methacrylic acid, and mixtures thereof, and for which the allyl ether unit comprising a fatty chain corresponds to the monomer of formula (XV):

CH₂═CR′CH₂OBₙR (XV)

wherein:

R′ is chosen from H and CH₃,

B is an ethyleneoxy radical,

n is an integer ranging from 0 to 100, and

R is a hydrocarbon radical chosen from alkyl, arylalkyl, aryl, alkylaryl, and cycloalkyl radicals comprising from 8 to 30 carbon atoms, for example, from 10 to 24, or from 12 to 18 carbon atoms. In at least one embodiment, in the unit of formula (XV), R′ is H, n is equal to 10, and R is a stearyl (C₁₈) radical.

Anionic associative polymers of this type are disclosed and prepared, according to an emulsion polymerization process, for example, in European Patent No. 0 216 479.

The anionic associative polymers may be chosen, in at least one embodiment, from polymers formed from 20 to 60% by weight of acrylic acid and/or methacrylic acid, from 5 to 60% by weight of lower alkyl (meth)acrylates, from 2 to 50% by weight of allyl ether comprising a fatty chain of formula (XV), and from 0 to 1% by weight of a crosslinking agent which may be chosen from conventional copolymerizable polyethylenic unsaturated monomers, such as diallyl phthalate, allyl (meth)acrylate, divinylbenzene, (poly)ethylene glycol dimethacrylate, and methylenebisacrylamide.

Additional examples of these polymers include, but are not limited to, crosslinked terpolymers of methacrylic acid, of ethyl acrylate and of polyethylene glycol (10 EO) ether of stearyl alcohol (Steareth 10), such as those sold by Allied Colloids under the names Salcare SC 80® and Salcare SC90®, which are 30% aqueous emulsions of a crosslinked terpolymer of methacrylic acid, of ethyl acrylate and of steareth-10 allyl ether (40/50/10).

(II) those comprising at least one hydrophilic unit of olefinic unsaturated carboxylic acid type and at least one hydrophobic unit of alkyl (C₁₀-C₃₀) ester of unsaturated carboxylic acid type.

These polymers may be chosen from those for which the hydrophilic unit of olefinic unsaturated carboxylic acid type is chosen from monomers of formula (XVI):

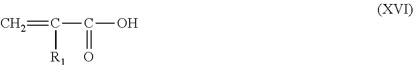

wherein R₁ is chosen from H, CH₃, and C₂H₅, i.e., acrylic acid, methacrylic acid, and ethacrylic acid units, and for which the hydrophobic unit of alkyl (C₁₀-C₃₀) ester of unsaturated carboxylic acid type is chosen from monomers of formula (XVII):

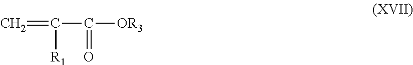

wherein R₂ is chosen from H, CH₃, and C₂H₅ (i.e., acrylate, methacrylate, and ethacrylate units), and in at least one embodiment, H (acrylate units) and CH₃ (methacrylate units), and R₃ is chosen from C₁₀-C₃₀, for example, C₁₂-C₂₂ alkyl radicals.

Alkyl (C₁₀-C₃₀) esters of unsaturated carboxylic acids in accordance with the present disclosure may comprise, for example, lauryl acrylate, stearyl acrylate, decyl acrylate, isodecyl acrylate, dodecyl acrylate, and the corresponding methacrylates, lauryl methacrylate, stearyl methacrylate, decyl methacrylate, isodecyl methacrylate, and dodecyl methacrylate.

Anionic polymers of this type are, for example, disclosed and prepared in U.S. Pat. Nos. 3,915,921 and 4,509,949.

In at least one embodiment, the anionic associative polymers may be chosen from polymers formed from a mixture of monomers comprising:

(i) essentially acrylic acid,

(ii) an ester of formula (XVII) described above and in which R₂ is chosen from H and CH₃, R₃ is an alkyl radical comprising from 12 to 22 carbon atoms, and

(iii) a crosslinking agent chosen from conventional copolymerizable polyethylenic unsaturated monomers, such as diallyl phthalate, allyl (meth)acrylate, divinylbenzene, (poly)ethylene glycol dimethacrylate, and methylenebisacrylamide.

According to another embodiment, the anionic associative polymers may be chosen from those comprising from 60 to 95% by weight of acrylic acid (hydrophilic unit), from 4 to 40% by weight of C₁₀-C₃₀ alkyl acrylate (hydrophobic unit), and from 0 to 6% by weight of crosslinking polymerizable monomer; and those comprising from 96 to 98% by weight of acrylic acid (hydrophilic unit), from 1 to 4% by weight of C₁₀-C₃₀ alkyl acrylate (hydrophobic unit) and from 0.1 to 0.6% by weight of crosslinking polymerizable monomer such as those described above.

Non-limiting examples of suitable commercial products include the products sold by Goodrich under the tradenames Pemulen TR1®, Pemulen TR2®, and Carbopol 1382®, and in at least one embodiment, Pemulen TR1® and the product sold by S.E.P.P.I.C. under the name Coatex SX®.

(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by Newphase Technologies.

(IV) acrylic terpolymers comprising:

(a) from 20% to 70% by weight of a carboxylic acid comprising α,β-monoethylenic unsaturation,

(b) from 20 to 80% by weight of a non-surface-active monomer comprising α,β-monoethylenic unsaturation other than (a),

(c) from 0.5 to 60% by weight of a nonionic monourethane which is the reaction product of a monohydric surfactant with a monoisocyanate comprising monoethylenic unsaturation,

such as those disclosed in European Patent Application No. 0 173 109, for example, disclosed in Example 3, i.e., a methacrylic acid/methyl acrylate/dimethyl(meta-isopropenyl)benzyl isocyanate terpolymer of ethoxylated behenyl alcohol (40 EO) as a 25% by weight aqueous dispersion.

(V) copolymers comprising, among their monomers, a carboxylic acid comprising α,β-monoethylenic unsaturation and an ester of a carboxylic acid comprising α,β-monoethylenic unsaturation and of an oxyalkylenated fatty alcohol.

In at least one embodiment, these compounds also comprise, as monomer, an ester of a carboxylic acid comprising α,β-monoethylenic unsaturation and of a C₁-C₄ alcohol.

An example of this type of compound includes, but is not limited to, Aculyn 22®, sold by Röhm & Haas, which is a methacrylic acid/ethyl acrylate/oxyalkylenated stearyl methacrylate terpolymer.

Cationic associative polymers include, for example:
(I) associative cationic polyurethanes, the family of which is disclosed, for example, in French Patent Application No. 00/09609; and represented by formula (XVIII):

R—X—(P)ₙ-[L-(Y)ₘ]ᵣ-L′-(P′)ₚ—X′—R′ (XVIII)

wherein:

R and R′, which may be identical or different, are chosen from hydrophobic groups and hydrogen;

X and X′, which may be identical or different, are chosen from groups comprising an amine functional group which may or may not carry a hydrophobic group, and L″ groups;

L, L′, and L″, which may be identical or different, are chosen from groups derived from a diisocyanate;

P and P′, which may be identical or different, are chosen from groups comprising an amine functional group which may or may not carry a hydrophobic group;

Y is a hydrophilic group;

r is an integer ranging from 1 to 100, for example, from 1 to 50, or from 1 to 25, and

n, m, and p, which may be identical or different, are numbers ranging from 0 to 1000;

wherein the molecule comprises at least one protonated or quaternized amine functional group and at least one hydrophobic group.

In at least one embodiment, the sole hydrophobic groups of these polyurethanes are the R and R′ groups at the chain ends.

Another non-limiting examples of a suitable family of cationic associative polyurethanes includes those of formula (XVIII) described above in which:
R and R′, which may be identical or different, are chosen from hydrophobic groups,

X and X′, which may be identical or different, are chosen from L″ groups,

n and p, which may be identical or different, are numbers ranging from 1 to 1,000, and

L, L′, L″, P, P′, Y, and m have the meanings indicated above.

Another non-limiting example of a suitable family of cationic associative polyurethanes includes those of formula (XVIII) above in which:
R and R′, which may be identical or different, are chosen from hydrophobic groups,

X and X′, which may be identical or different, are chosen from L″ groups,

n and p are equal to 0, and

L, L′, L″, Y and m have the meanings indicated above.

The fact than n and p have the value 0 means that these polymers do not comprise units derived from a monomer comprising an amine functional group incorporated in a polymer during the polycondensation. The protonated amine functional groups of these polyurethanes result from the hydrolysis of isocyanate functional groups, in excess, at the chain end, followed by the alkylation of the primary amine functional groups formed by alkylating agents comprising a hydrophobic group, that is to say compounds of RQ or R′Q type in which R and R′ are as defined above and Q is a leaving group, such as a halide, a sulphate, and the like.

Yet another non-limiting example of a suitable family of cationic associative polyurethanes is that corresponding to the formula (XVIII) above in which:

R and R′, which may be identical or different, are chosen from hydrophobic groups,

X and X′, which may be identical or different, are chosen from groups comprising a quaternary amine,

n and p are equal to zero, and

L, L′, Y, and m have the meanings indicated above.

The number-average molecular weight of the cationic associative polyurethanes may range from 400 to 500,000, for example, from 1,000 to 400,000, or from 1,000 to 300,000.

As used herein, the term “hydrophobic group” is understood to mean a radical or polymer comprising a saturated or unsaturated and linear or branched hydrocarbon chain, which can comprise at least one heteroatom, such as P, O, N, and S, or a radical comprising a chain chosen from perfluorinated and silicone chains. When it denotes a hydrocarbon radical, the hydrophobic group comprises at least 10 carbon atoms, for example, from 10 to 30 carbon atoms, from 12 to 30 carbon atoms, or from 18 to 30 carbon atoms.

According to one embodiment, the hydrocarbon group originates from one monofunctional compound.

For example, the hydrophobic group may result from a fatty alcohol, such as stearyl alcohol, dodecyl alcohol, and decyl alcohol. It may also denote a hydrocarbon polymer, for example, polybutadiene.

When X and/or X′ are groups comprising a tertiary or quaternary amine, X and/or X′ may be chosen from groups of the following formulae:

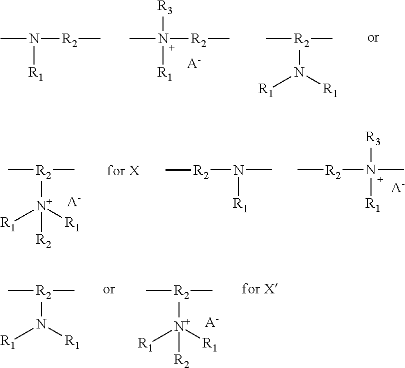

wherein:

R₂ is chosen from linear or branched alkylene radicals comprising from 1 to 20 carbon atoms, which may or may not comprise a saturated or unsaturated ring, and arylene radicals, it being possible for at least one of the carbon atoms to be replaced by a heteroatom chosen from N, S, O, and P;

R₁ and R₃, which may be identical or different, are chosen from linear and branched C₁-C₃₀ alkyl and alkenyl radicals and aryl radicals, it being possible for at least one of the carbon atoms to be replaced by a heteroatom chosen from N, S, O, and P; and

A⁻ is a physiologically acceptable counterion.

L, L′, and L″ are chosen from groups of formula:

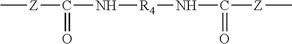

wherein:

Z is chosen from —O—, —S—, and —NH—; and

R₄ is chosen from linear and branched alkylene radicals comprising from 1 to 20 carbon atoms, which may or may not comprise a saturated or unsaturated ring, and arylene radicals, it being possible for at least one of the carbon atoms to be replaced by a heteroatom chosen from N, S, O, and P.

The P and P′ groups, comprising an amine functional group, may be chosen from groups of the following formulae:

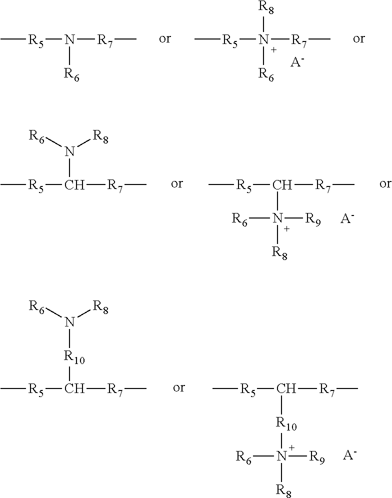

wherein:

R₅ and R₇ have the same meanings as H₂ defined above;

R₆, R₈ and R₉ have the same meanings as R₁ and R₃ defined above;

R₁₀ is chosen from linear and branched alkylene groups which are optionally unsaturated and which may comprise at least one heteroatom chosen from N, O, S, and P, and

A⁻ is a physiologically acceptable counterion.

With respect to the meaning for Y, the term “hydrophilic group” is understood to mean a water-soluble polymeric or nonpolymeric group.

For example, when polymers are not concerned, examples include ethylene glycol, diethylene glycol, and propylene glycol.

When, in accordance with at least one embodiment, the hydrophilic polymer is concerned, non-limiting examples include polyethers, sulphonated polyesters, sulphonated polyamides, and blends thereof. According to another embodiment, the hydrophilic compound is a polyether, for instance, poly(ethylene oxides) and poly(propylene oxides).

The cationic associative polyurethanes of formula (XVIII) which may be used according to the present disclosure may be formed from diisocyanates and from various compounds having functional groups comprising a labile hydrogen. The functional groups comprising a labile hydrogen may be chosen from alcohol, primary or secondary amine groups, and primary or secondary thiol functional groups which give, after reaction with the diisocyanate functional groups, polyurethanes, polyureas, and polythioureas respectively. As used herein, the term “polyurethanes” which can be used according to the present disclosure encompasses these three types of polymers, namely polyurethanes, polyureas, and polythioureas, and copolymers of these.

A first type of compound useful in the preparation of the polyurethane of formula (XVIII) is a compound comprising at least one unit comprising an amine functional group. This compound can be polyfunctional, and in at least one embodiment, the compound is difunctional, that is to say, this compound comprises two labile hydrogen atoms carried, for example, by a group chosen from hydroxyl, primary amine, secondary amine, and thiol functional groups. Use may also be made of a mixture of polyfunctional and difunctional compounds in which the percentage of polyfunctional compounds is low.

As indicated above, this compound may comprise more than one unit comprising an amine functional group. It is then a polymer carrying a repetition of the unit comprising an amine functional group.

Compounds of this type can be represented by one of the following formulae:

HZ—(P)ₙ—ZH,

or

HZ—(P′)ₚ—ZH

wherein Z, P, P′, n, and p are as defined above.

Examples of compound comprising an amine functional group include, but are not limited to, N-methyldiethanolamine, N-tert-butyldiethanolamine, and N-sulphoethyldiethanolamine.

A second compound useful in the preparation of the polyurethane of formula (XVIII) is a diisocyanate corresponding to the formula:

O═C═N—R₄—N═C═O

wherein R₄ is as defined above.

Non-limiting examples of this second compound include methylenediphenyl diisocyanate, methylenedicyclohexane diisocyanate, isophorone diisocyanate, toluene diisocyanate, naphthalene diisocyanate, butane diisocyanate, and hexane diisocyanate.

A third compound useful in the preparation of the polyurethane of formula (XVIII) is a hydrophobic compound intended to form the end hydrophobic groups of the polymer of formula (XVIII).

This compound comprises a hydrophobic group and a functional group comprising a labile hydrogen, for example hydroxyl, and primary and secondary amine and thiol functional groups.

For example, this compound can be a fatty alcohol, such as stearyl alcohol, dodecyl alcohol, and decyl alcohol. When this compound comprises a polymer chain, it may, for example, be α-hydroxyl hydrogenated polybutadiene.

The hydrophobic group of the polyurethane of formula (XVIII) may also result from the quaternization reaction of the tertiary amine of the compound comprising at least one tertiary amine unit. Thus, the hydrophobic group is introduced by the quaternizing agent. This quaternizing agent may be a compound of RQ or R′Q type in which R and R′ are as defined above and Q is a leaving group, such as a halide, a sulphate, and the like.

The cationic associative polyurethane may additionally comprise a hydrophilic sequence. This sequence is contributed by a fourth type of compound participating in the preparation of the polymer. In at least one embodiment, this compound may be polyfunctional, for example, difunctional. It is also possible to use a mixture where the percentage of polyfunctional compound is low.

The functional groups comprising a labile hydrogen may be chosen from alcohol groups, primary and secondary amine groups, and primary and secondary thiol functional groups. This compound can be, for example, a polymer terminated at the ends of the chains by one of these functional groups comprising a labile hydrogen.

For example, when polymers are not concerned, ethylene glycol, diethylene glycol, and propylene glycol may be used.

When a hydrophilic polymer is concerned, polyethers, sulphonated polyesters, sulphonated polyamides, and blends thereof may be used. In at least one embodiment, the hydrophilic compound is a polyether, for example, poly(ethylene oxides) and poly(propylene oxides).

The hydrophilic group Y in the formula (XVIII) is optional. This is because the units comprising a quaternary or protonated amine functional group may be sufficient to contribute to the solubility or water-dispersibility necessary for this type of polymer in an aqueous solution.

Although the presence of a hydrophilic Y group is optional, in at least one embodiment, the cationic associative polyurethanes do comprise such a group.
(II) quaternized cellulose derivatives and polyacrylates comprising noncyclic aminated side groups.

The quaternized cellulose derivatives include, for example:

quaternized celluloses modified by groups comprising at least one fatty chain, such as alkyl, arylalkyl, and alkylaryl groups comprising at least 8 carbon atoms, and mixtures thereof, and

quaternized hydroxyethylcelluloses modified by groups comprising at least one fatty chain, such as alkyl, arylalkyl, and alkylaryl groups comprising at least 8 carbon atoms, and mixtures thereof.

The alkyl radicals carried by the above quaternized celluloses or hydroxyethylcelluloses may comprise, for example, from 8 to 30 carbon atoms. The aryl radicals may be chosen, for example, from phenyl, benzyl, naphthyl, and anthryl groups.

Examples of commercially available quaternized alkylhydroxyethylcelluloses comprising C₈-C₃₀ fatty chains include, but are not limited to, the products Quatrisoft LM 200®, Quatrisoft LM-X 529-18-A®, Quatrisoft LM-X 529-18B® (C₁₂ alkyl), and Quatrisoft LM-X 529-8® (C₁₈ alkyl) sold by Amerchol and the products Crodacel QM®, Crodacel QL® (C₁₂ alkyl), and Crodacel QS® (C₁₈ alkyl) sold by Croda.

The amphoteric associative polymers may be chosen, for example, from those comprising at least one noncyclic cationic unit. For instance, the polymers may be chosen from those prepared from or comprising from 1 to 20 mol % of monomer comprising a fatty chain, for example, from 1.5 to 15 mol %, or from 1.5 to 6 mol %, with respect to the total number of moles of monomers.

The amphoteric associative polymers according to the present disclosure, in at least one embodiment, comprise or are prepared by copolymerizing:

1) at least one monomer chosen from those of formula (XIX) and (XX):

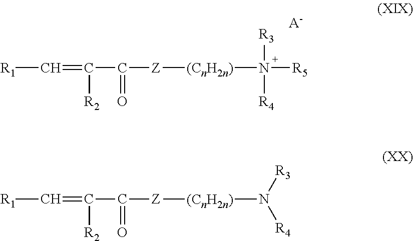

wherein:

R₁ and R₂, which may be identical or different, are chosen from hydrogen and methyl radicals,

R₃, R₄ and R₅, which may be identical or different, are chosen from linear and branched alkyl radicals comprising from 1 to 30 carbon atoms,

Z is chosen from NH and oxygen,

n is an integer ranging from 2 to 5, and

A⁻ is an anion resulting from an organic or inorganic acid, such as methyl sulphate anions and halides such as chloride and bromide;

2) at least one monomer of formula (XXI):

R₆—CH═CR₇—COOH (XXI)

wherein R₆ and R₇, which may be identical or different, are chosen from hydrogen and methyl radicals; and

3) at least one monomer of formula (XXII):

R₆—CH═CR₇—COXR₈ (XXII)

wherein R₆ and R₇, which may be identical or different, are chosen from hydrogen and methyl radicals, X is chosen from oxygen and nitrogen, and R₈ is chosen from linear and branched alkyl radicals comprising from 1 to 30 carbon atoms;

wherein at least one of the monomers of formulae (XIX), (XX), and (XXII) comprises at least one fatty chain.

The monomers of formulae (XIX) and (XX) of the present disclosure may be chosen, for example, from:

dimethylaminoethyl methacrylate, dimethylaminoethyl acrylate,

diethylaminoethyl methacrylate, diethylaminoethyl acrylate,

dimethylaminopropyl methacrylate, dimethylaminopropyl acrylate,

dimethylaminopropylmethacrylamide, and dimethylaminopropylacrylamide,

these monomers optionally being quaternized, for example by a C₁-C₄ alkyl halide or a di(C₁-C₄ alkyl) sulphate.

In another embodiment, the monomer of formula (XIX) may be chosen from acrylamidopropyltrimethylammonium chloride and methacrylamidopropyltrimethylammonium chloride.

The monomers of formula (XXI) of the present disclosure may be chosen from acrylic acid, methacrylic acid, crotonic acid, and 2-methylcrotonic acid. In at least one embodiment, the monomer of formula (XXI) is acrylic acid.

The monomers of formula (XXII) of the present disclosure may be chosen, for instance, from C₁₂-C₂₂, such as C₁₆-C₁₈, alkyl acrylates and methacrylates.

The monomers constituting the amphoteric polymers comprising a fatty chain of the present disclosure are, according to one embodiment, neutralized and/or quaternized.

In another embodiment, the ratio of the number of cationic charges to the number of anionic charges can be equal to approximately 1.

The amphoteric associative polymers according to the present disclosure can comprise from 1 to 10 mol % of the monomer comprising a fatty chain (i.e, monomers of formulas (XIX), (XX), and (XXII)), for example, from 1.5 to 6 mol %, with respect to the total number of moles of monomers.

The weight-average molecular weights of the amphoteric associative polymers according to the present disclosure may range from 500 to 50,000,000, for example, from 10,000 to 5,000,000.

The amphoteric associative polymers according to the present disclosure can also comprise other monomers, such as nonionic monomers, for example, C₁-C₄ alkyl acrylates and methacrylates.

Amphoteric associative polymers according to the present disclosure may include, for example, those disclosed and prepared in International Patent Application Publication No. WO 98/44012.

The amphoteric associative polymers may, for example, be chosen from acrylic acid/(meth)acrylamidopropyltrimethylammonium chloride/stearyl methacrylate terpolymers.

The nonionic associative polymers may be chosen, for instance, from:

(1) celluloses modified by groups comprising at least one fatty chain; for example:
hydroxyethylcelluloses modified by groups comprising at least one fatty chain, such as alkyl, arylalkyl, and alkylaryl groups, and mixtures thereof, and in which the alkyl groups may be chosen from C₈-C₂₂ alkyl groups, such as the product Natrosol Plus Grade 330 CS® (C₁₆ alkyl) sold by Aqualon and the product Bermocoll EHM 100® sold by Berol Nobel,
those modified by alkylphenol polyalkylene glycol ether groups, such as the product Amercell Polymer HM-1500® (nonylphenol polyethylene glycol (15) ether) sold by Amerchol.

(2) hydroxypropyl guars modified by groups comprising at least one fatty chain, such as the product Esaflor HM 22® (C₂₋₂ alkyl chain) sold by Lamberti and the products RE210-18® (C₁₄ alkyl chain) and RE205-1® (C₂₀ alkyl chain) sold by Rhone-Poulenc.

(3) copolymers of vinylpyrrolidone and of hydrophobic monomers comprising a fatty chain, for example:
the products Antaron V216@ and Ganex V2160 (vinyl-pyrrolidone/hexadecene copolymer) sold by I.S.P.,
the products Antaron V220® and Ganex V220® (vinylpyrrolidone/eicosene copolymer) sold by I.S.P.

(4) copolymers of C₁-C₆ alkyl methacrylates and acrylates and of amphiphilic monomers comprising at least one fatty chain, for example, the methyl acrylate/oxyethylenated stearyl acrylate copolymer sold by Goldschmidt under the name Antil 208®.

(5) copolymers of hydrophilic methacrylates and acrylates and of hydrophobic monomers comprising at least one fatty chain, for example, the polyethylene glycol methacrylate/lauryl methacrylate copolymer.

(6) polyether polyurethanes comprising, in their chain, both hydrophilic sequences of generally polyoxyethylene nature and hydrophobic sequences which can be aliphatic strings only and/or cycloaliphatic and/or aromatic strings.

(7) polymers comprising an aminoplast ether backbone having at least one fatty chain, such as the compounds Pure Thix® provided by Sud-Chemie.

In at least one embodiment, the polyether polyurethanes comprise at least two lipophilic hydrocarbon chains comprising from 6 to 30 carbon atoms which are separated by a hydrophilic sequence, it being possible for the hydrocarbon chains to be pendant chains or chains at the end of the hydrophilic sequence. In another embodiment, it is possible for at least one hydrocarbon chain to be a pendant chain. In addition, the polymer may comprise a hydrocarbon chain at one end or at both ends of a hydrophilic sequence.

The polyether polyurethanes can be polysequential, for example, in the triblock form. The hydrophobic sequences can be at each end of the chain (for example: triblock copolymer comprising a central hydrophilic sequence) or distributed both at the ends and in the chain (for example: polysequential copolymer). These polymers may also be chosen from graft polymers and star polymers.

The nonionic polyether polyurethanes comprising a fatty chain can be triblock copolymers, the hydrophilic sequence of which is a polyoxyethylene chain comprising from 50 to 1,000 oxyethylene groups. The nonionic polyether polyurethanes comprise a urethane bond between the hydrophilic sequences, hence the origin of the name.

By extension, the nonionic polyether polyurethanes comprising a fatty chain may optionally include those, the hydrophilic sequences of which are bonded to the lipophilic sequences via other chemical bonds.

Further examples of nonionic polyether polyurethanes comprising a fatty chain which can be used in accordance with the present disclosure include Rhéolate 205® comprising a urea functional group sold by Rheox, Rhéolates® 208, 204, and 212, Acrysol RM 184®, Elfacos T210® comprising a C₁₂₋₁₄ alkyl chain, Elfacos T212® comprising a C₁₈ alkyl chain from Akzo, and DW 1206B® from Röhm & Haas comprising a C₂₀ alkyl chain and comprising a urethane bond, provided at a dry matter content of 20% in water.

Use may also be made of solutions or dispersions of these polymers, for example, in a medium chosen from water and aqueous/alcoholic mediums. Examples of such polymers include, for example, Rhéolate® 255, Rhéolate® 278, and Rhéolate® 244, sold by Rheox and DW 1206F and DW 1206J provided by Röhm & Haas.

The polyether polyurethanes which may be used according to the present disclosure include those described in the paper by G. Formum, J. Bakke and Fk. Hansen, Colloid Polym. Sci., 271, 380-389 (1993).

In yet another embodiment, the polyether polyurethane may be chosen from those capable of being obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

Such polyether polyurethanes are sold, for example, by Röhm & Haas under the names Aculyn 46® and Aculyn 44® (Aculyn 46® is a polycondensate of polyethylene glycol comprising 150 or 180 mol of ethylene oxide, of stearyl alcohol and of methylenebis(4-cyclohexyl isocyanate) (SMDI), at 15% by weight in a matrix of maltodextrin (4%) and of water (81%); Aculyn 44® is a polycondensate of polyethylene glycol comprising 150 or 180 mol of ethylene oxide, of decyl alcohol and of methylenebis(4-cyclohexyl isocyanate) (SMDI), at 35% by weight in a mixture of propylene glycol (39%) and of water (26%)).

According to another embodiment, the composition according to the present disclosure comprises at least one anionic associative polymer, present in an amount ranging from 0.1 to 5%, for example, from 0.3 to 3%, by weight, with respect to the total weight of the composition.

The composition may also comprise at least one additive chosen from cosmetic adjuvants and active principles commonly used in the hair field. These additives may be chosen, for example, from vitamins, amino acids, oligopeptides, peptides, hydrolysed or nonhydrolysed and modified or unmodified proteins, enzymes, branched or unbranched fatty acids and alcohols, animal, vegetable, or mineral waxes, ceramides, pseudoceramides, hydroxylated organic acids, UV screening agents, antioxidants and agents for combating free radicals, chelating agents, antidandruff agents, seborrhoea-regulating agents, soothing agents, ionic or nonionic surface-active agents, silicones, mineral, vegetable, or animal oils, polyisobutenes and poly(α-olefin)s, additional fatty esters other than the esters of polyethylene glycol and the fatty acid mentioned above, hair coloring agents, such as direct dyes, oxidation dye precursors, and pigments, acids, bases, plasticizers, fragrances, preservatives, inorganic fillers, pearlescent agents, and glitter.

These additives may be present in the composition according to the present disclosure in an amount ranging from 0 to 20% by weight, with respect to the total weight of the composition.

Of course, a person skilled in the art will take care to choose the at least one additional cosmetic adjuvant and/or active principle so that the beneficial properties intrinsically attached to the arrangement and process in accordance with the present disclosure are not, or not substantially, detrimentally affected by the envisaged addition or additions.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLES

The following compositions (as % of active material) were prepared):

TABLE 1

| INCI name | Ex. 1 | Ex. 2 | Ex. 3 | Ex. 4 | Ex. 5 |
|---|---|---|---|---|---|
| Polyquaternium-11 | 6% | | | | |
| VA/Vinyl | | 7% | 10% | | |
| butylbenzoate/Crotonates | | | | | |
| copolymer | | | | | |
| Polyquaternium-4 | | | | 1.5% | |
| AMP Acrylates/Allyl | | | | | 6% |
| methacrylate copolymer | | | | | |
| PEG-100 Stearate | 3% | | | | 1.5% |
| PEG-30 Glyceryl | | | | 5% | |
| Cocoate | | | | | |
| PEG-150 Distearate | | 2.5% | 3% | 1% | 5% |
| PEG-200 Glyceryl | 2% | | 3% | | |
| Stearate | | | | | |
| Glycerol | 5% | 3% | | | |
| Propylene Glycol | | | 5% | 3% | 3% |
| Hydroxypropyl guar | | | | 1% | |
| Sepigel 305 | 1.5% | 0.6% | 1% | | |
| (acrylamidopropane- | | | | | |
| sulphonic | | | | | |
| acid polymer as inverse | | | | | |
| emulsion) | | | | | |
| Carbomer | | | | 0.6% | 0.2% |
| PEG-40 Hydrogenated | 1% | 0.5% | 1% | 0.5% | 0.5% |
| Castor Oil | | | | | |
| Ethanol | | 6% | | | |
| Preservatives, | q.s. | q.s. | q.s. | q.s. | q.s. |
| Neutralizing | | | | | |
| agent, Fragrance | | | | | |
| Water | q.s. for | q.s. for | q.s. for | q.s. for | q.s. for |
| | 100 | 100 | 100 | 100 | 100 |

TABLE 2

| INCI name | Ex. 6 | Ex. 7 | Ex. 8 |
|---|---|---|---|
| Carbomer | 1.4% | 0.4% | 0.1% |
| Polyquaternium-11 | 4% | | |
| VA/Vinyl | | | |
| butylbenzoate/Crotonates | | | |
| copolymer | | | |
| Polyquaternium-4 | | 2% | |
| AMP Acrylates/Allyl | | | 5% |
| methacrylate copolymer | | | |
| PEG-30 Glyceryl Cocoate | | 2% | |
| PEG-150 Distearate | 3% | 2% | |
| PEG-200 Glyceryl Stearate | | | 5% |
| Glycerol | 5% | | |
| Propylene Glycol | | 3% | 1% |
| Hydroxypropyl guar | | 0.5% | |
| PEG-40 Hydrogenated Castor | 0.5% | 0.5% | 0.5% |
| Oil | | | |
| Ethanol | | | 2% |
| Preservatives, Neutralizing | q.s. | q.s. | q.s. |
| agent, Fragrance | | | |
| Water | q.s. for | q.s. for | q.s. for |
| | 100 | 100 | 100 |

These compositions exhibited good fixing properties and also persistence of these fixing properties over time.

1-22. (canceled)

23. A non-washing cosmetic composition comprising, in a cosmetically acceptable medium, at least one anionic fixing polymer, at least one ester of polyethylene glycol and of fatty acid, and at least one thickening agent;
wherein the cosmetically acceptable medium is chosen from alcoholic and aqueous/alcoholic mediums;
wherein the at least one ester of polyethylene glycol and fatty acid is present in the composition in an amount ranging from 1% to 10% by weight relative to the total weight of the composition;
wherein at least one thickening agent is chosen from coconut diethanolamide, coconut monoethanolamide, oxyethylenated alkyl ether carboxylic acid monoethanolamide, hydroxyethylcellulose, hydroxypropylcellulose, carboxymethylcellulose, hydroxypropyl guar, xanthan gum, scleroglucan gum, crosslinked homopolymers of acrylic acid, copolymers of acrylic acid, crosslinked homopolymers of acrylamidopropanesulphonic acid, copolymers of acrylamidopropanesulphonic acid, and associative thickening polymers;
wherein the at least one ester of polyethylene glycol and fatty acid is chosen from those of formula:
R₁CO—(OCH₂CH₂)ₙ₀-[OCH₂—CH(OR₂)—CH₂]ₙ₁—(OCH₂CH₂)ₙ₂—R₃
wherein
R₂ is chosen from hydrogen and (CH₂CH₂O)ₙ₃COR₄ groups;
n1 is an integer equal to 0 or 1;
n2 is an integer ranging from 2 to 300;
n3 is an integer ranging from 1 to 300;
n0 is an integer ranging from 0 to 300;
R₃ is chosen from OH groups and R₅COO groups;
R₁, R₄, and R₅, which may be identical or different, are chosen from C₁₀ to C₃₀ alkyl groups and C₁₀ to C₃₀ alkylene groups.

24. The cosmetic composition of claim 23, wherein the at least one anionic fixing polymer is chosen from homopolymers and copolymers of acrylic and methacrylic acid and the salts thereof, crotonic acid copolymers, copolymers of monounsaturated C₄-C₈ carboxylic acids and anhydrides, polyacrylamides comprising carboxylate groups, homopolymers and copolymers comprising sulphonic groups, anionic polyurethanes, and grafted anionic silicone polymers.

25. The cosmetic composition of claim 23, further comprising at least one fixing polymer chosen from cationic and amphoteric fixing polymers.

26. The cosmetic composition of claim 25, wherein the at least one cationic fixing polymer is chosen from homopolymers and copolymers of acrylic and methacrylic esters and amides comprising aminated functional groups, cationic guar gums, quaternary copolymers of vinylpyrrolidone and of vinylimidazole, and chitosans.

27. The cosmetic composition of claim 25, wherein the at least one amphoteric fixing polymer is chosen from copolymers comprising acidic vinyl units and comprising basic vinyl units, crosslinked and alkylated polyaminoamides, polymers comprising zwitterionic units, polymers derived from chitosan, modified (C₁-C₅)alkyl vinyl ether/maleic anhydride copolymers, amphoteric polyurethanes, and amphoteric grafted silicone polymers.

28. The cosmetic composition of claim 23, wherein the at least one anionic fixing polymer is present in an amount ranging from 0.1 to 20% by weight relative to the total weight of the composition.

29. The cosmetic composition of claim 28, wherein the at least one anionic fixing polymer is present in an amount ranging from 1 to 15% by weight relative to the total weight of the composition.

30. The cosmetic composition of claim 1, wherein the at least one thickening agent is present in the composition in an amount ranging from 0.01% to 10% by weight relative to the total weight of the composition.

31. The cosmetic composition of claim 16, wherein the at least one thickening agent is present in the composition in an amount ranging from 0.3% to 3% by weight relative to the total weight of the composition.

32. The cosmetic composition of claim 1, further comprising at least one additive chosen from vitamins; amino acids; oligopeptides; peptides; hydrolysed and nonhydrolysed, modified and unmodified proteins; enzymes; branched and unbranched fatty acids and alcohols; animal, vegetable, and mineral waxes; ceramides and pseudoceramides; hydroxylated organic acids; UV screening agents; antioxidants and agents for combating free radicals; chelating agents; antidandruff agents; seborrhoea-regulating agents; soothing agents; ionic and nonionic surface-active agents; silicones; mineral, vegetable, and animal oils; polyisobutenes and poly(α-olefin)s; additional fatty esters; hair coloring agents; acids; bases; plasticizers; fragrances; preservatives; inorganic fillers; pearlescent agents; and glitter.

33. The cosmetic composition of claim 1, wherein the composition is packaged such that it is provided in the form of a spray.

34. The cosmetic composition of claim 1, provided in the form of a gel.

35. The cosmetic composition of claim 1, wherein the composition is packaged such that it is provided in the form of a foam.

36. A method for shaping and/or promoting the form retention of a hairstyle, and/or conferring good hold over time on a hairstyle, comprising applying a cosmetic composition to the hair,
wherein the cosmetic composition comprises, in a cosmetically acceptable medium, at least one anionic fixing polymer, at least one ester of polyethylene glycol and of fatty acid, and at least one thickening agent; and
wherein the cosmetically acceptable medium is chosen from alcoholic and aqueous/alcoholic mediums;
wherein the at least one ester of polyethylene glycol and fatty acid is present in the composition in an amount ranging from 1% to 10% by weight relative to the total weight of the composition;
wherein at least one thickening agent is chosen from coconut diethanolamide, coconut monoethanolamide, oxyethylenated alkyl ether carboxylic acid monoethanolamide, hydroxyethylcellulose, hydroxypropylcellulose, carboxymethylcellulose, hydroxypropyl guar, xanthan gum, scleroglucan gum, crosslinked homopolymers of acrylic acid, copolymers of acrylic acid, crosslinked homopolymers of acrylamidopropanesulphonic acid, copolymers of acrylamidopropanesulphonic acid, and associative thickening polymers;
wherein the at least one ester of polyethylene glycol and fatty acid is chosen from those of formula:
R₁CO—(OCH₂CH₂)ₙ₀-[OCH₂—CH(OR₂)—CH₂]ₙ₁—(OCH₂CH₂)ₙ₂—R₃
wherein
R₂ is chosen from hydrogen and (CH₂CH₂O)ₙ₃COR₄ groups;
n1 is an integer equal to 0 or 1;
n2 is an integer ranging from 2 to 300;
n3 is an integer ranging from 1 to 300;
n0 is an integer ranging from 0 to 300;
R₃ is chosen from OH groups and R₅COO groups;
R₁, R₄, and R₅, which may be identical or different, are chosen from C₁₀ to C₃₀ alkyl groups and C₁₀ to C₃₀ alkylene groups.
